SEQUENCE LISTING (35 sequences)

SEQ ID NO: 1 - protein, 16 aa, Artificial
  Synthetic peptide
NQFLPYPYYAKPAAVR

SEQ ID NO: 2 - protein, 16 aa, Artificial
  Synthetic peptide
STEVFTKKTKLTEEEK

SEQ ID NO: 3 - protein, 16 aa, Artificial
  Synthetic peptide
EKNRLNFLKKISQRYQ

SEQ ID NO: 4 - protein, 16 aa, Artificial
  Synthetic peptide
YQLDAYPSGAWYYVPL

SEQ ID NO: 5 - protein, 16 aa, Artificial
  Synthetic peptide
SSEEIVPNSVEQKHIQ

SEQ ID NO: 6 - protein, 16 aa, Artificial
  Synthetic peptide
HSMKEGIHAQQKEPMI

SEQ ID NO: 7 - protein, 16 aa, Artificial
  Synthetic peptide
INPSKENLCSTFCKEV

SEQ ID NO: 8 - protein, 16 aa, Artificial
  Synthetic peptide
QRYQKFALPQYLKTVY

SEQ ID NO: 9 - protein, 16 aa, Artificial
  Synthetic peptide
ESPPEINTVQVTSTAV

SEQ ID NO: 10 - protein, 16 aa, Artificial
  Synthetic peptide
QYTDAPSFSDIPNPIG

SEQ ID NO: 11 - protein, 16 aa, Artificial
  Synthetic peptide
LEIVPNSAEERLHSMK

SEQ ID NO: 12 - protein, 16 aa, Artificial
  Synthetic peptide
NLLRFFVAPFPEVFGK

SEQ ID NO: 13 - protein, 16 aa, Artificial
  Synthetic peptide
LNEINQFYQKFPQYLQ

SEQ ID NO: 14 - protein, 16 aa, Artificial
  Synthetic peptide
ESTEVFTKKTKLTEEE

SEQ ID NO: 15 - protein, 16 aa, Artificial
  Synthetic peptide
EKNRLNFLKKISQRYQ

SEQ ID NO: 16 - protein, 16 aa, Artificial
  Synthetic peptide
PLTQTPVVVPPFLQPE

SEQ ID NO: 17 - protein, 16 aa, Artificial
  Synthetic peptide
VENLHLPLPLLQSWMH

SEQ ID NO: 18 - protein, 16 aa, Artificial
  Synthetic peptide
RELEELNVPGEIVESL

SEQ ID NO: 19 - protein, 16 aa, Artificial
  Synthetic peptide
HKEMPFPKYPVEPFTE

SEQ ID NO: 20 - protein, 16 aa, Artificial
  Synthetic peptide
TQSLVYPFPGPIPNSL

SEQ ID NO: 21 - protein, 16 aa, Artificial
  Synthetic peptide
HQPLPPTVMFPPQSVL

SEQ ID NO: 22 - protein, 16 aa, Artificial
  Synthetic peptide
YIPIQYVLSRYPSYGL

SEQ ID NO: 23 - protein, 16 aa, Artificial
  Synthetic peptide
NNQFLPYPYYAKPAAV

SEQ ID NO: 24 - protein, 16 aa, Artificial
  Synthetic peptide
RCEKDERFFSDKIAKY

SEQ ID NO: 25 - protein, 16 aa, Artificial
  Synthetic peptide
VRSPAQILQWQVLSNT

SEQ ID NO: 26 - protein, 16 aa, Artificial
  Synthetic peptide
HPHLSFMAIPPKKNQD

SEQ ID NO: 27 - protein, 16 aa, Artificial
  Synthetic peptide
TEAVESTVATLEDSPE

SEQ ID NO: 28 - protein, 16 aa, Artificial
  Synthetic peptide
RELKDLKGYGGVSLPE

SEQ ID NO: 29 - protein, 16 aa, Artificial
  Synthetic peptide
EQLTKCEVFRELKDLK

SEQ ID NO: 30 - protein, 16 aa, Artificial
  Synthetic peptide
DIMCVKKILDKVGINY

SEQ ID NO: 31 - protein, 16 aa, Artificial
  Synthetic peptide
TKIPAVFKIDALNENK

SEQ ID NO: 32 - protein, 16 aa, Artificial
  Synthetic peptide
EVDDEALEKFDKALKA

SEQ ID NO: 33 - protein, 16 aa, Artificial
  Synthetic peptide
FDKALKALPMHIRLSF

SEQ ID NO: 34 - protein, 16 aa, Artificial
  Synthetic peptide
AQKKIIAEKTKIPAVF

SEQ ID NO: 35 - protein, 16 aa, Artificial
  Synthetic peptide
YTDAPSFSDIPNPIGS

(19) United States
(12) Patent Application Publication
(10) Pub. No.: US 2011/0046015 A1
(43) Pub. Date: Feb. 24, 2011

(54) PEPTIDE IMMOBILIZATION SOLUTION AND USE THEREOF

(76) Inventors: Hiroyuki Honda, Aichi (JP); Tsutomu Kawabe, Aichi (JP); Mina Okochi, Aichi (JP); Miyoko Matsushima, Aichi (JP); Naoki Matsumoto, Aichi (JP); Mitsuo Kawase, Aichi (JP); Yasuko Yoshida, Aichi (JP); Tomokazu Takase, Aichi (JP)

Correspondence Address: Tomoko Nakajima;Cermak Nakajima LLP, 127 S. Peyton Street, Suite 210, Alexandria, VA, 22314, US

(21) Appl. No.: 12/864,619

(22) PCT Filed: Jan. 29, 2009
(86) PCT No.: PCT/JP2009/051504

§ 371 Date: Oct. 27, 2010

(30) Foreign Application Priority Data
Jan. 29, 2008 (JP) 2008-018006
Sep. 24, 2008 (JP) 2008-243701

(51) Int. Cl. C40B 40/10; C07K 1/00; C07K 2/00; C09K 3/00

(52) U.S. Cl. 506/18; 530/345; 530/300; 252/182.12

(57) ABSTRACT

Disclosed is a peptide immobilization technique which can achieve the immobilization of a satisfactory quantity of a peptide on a solid support. In the immobilization of a peptide on a solid support, a surfactant is allowed to coexist on the solid support together with the peptide. In this manner, the quantity of the peptide immobilized on the solid support can be increased.

### BACKGROUND OF THE INVENTION

1. Field of the Invention

The present invention relates to a solution for immobilization of peptide and the use thereof and more particularly, to a peptide immobilization solution, a production method of a solid phase support on which peptide is immobilized, a peptide any and a method of using a peptide array.

2. Description of the Related Art

Proteins and peptides have attracted attention in recent years with respect to the mechanism of diseases. Although information relating to disease-related genes, their synthesis products and the proteins that they encode has come to be obtained through genome analyses, it is necessary to analyze the functions of proteins during actual diagnosis and drug development. In addition, in order to analyze various mechanisms of occurrence and utilize them in diagnosis and treatment, information is required regarding protein-mediated intracellular signal transduction. Thus, comprehensive analyses of proteins and peptides are required.

Although comprehensive analyses relating to DNA and other nucleic acids have already been realized through the use of DNA microarrays and the like, in addition to it being difficult to acquire comprehensive information regarding proteins, it is extremely difficult to immobilize proteins while maintaining their physiological activity. On the other hand, peptides, which are smaller molecules than proteins, can be chemically synthesized more easily than proteins, and are thought to present less of a problem of denauration than proteins.

A known example of this type of peptide army immobilizes peptides with an immobilization solution containing an organic solvent and DMSO (Tapia, V., et al., Anal. Biochem., 363, 108-118 (2007)). In addition to being used to search for substrates, ligands and inhibitors relating to intracellular signal transduction, protein arrays are also expected to be applied to searches for allergens and novel physiologically active peptides.

### SUMMARY OF THE INVENTION

In order to immobilize peptides and search for various actions thereof, it is necessary to construct a highly sensitive and highly reliable evaluation system. For example, in the case of forming peptides into an array, it is required to immobilize a copious amount of peptides as possible in individual evaluation areas (typically, spots) on a solid phase support, and inhibit variations in the amounts of peptide immobilized between the individual evaluation areas. In actuality, however, according to the inventors of the present invention, the amount of peptide immobilized on a solid phase support per evaluation area was determined to be not always adequate, and variations in immobilized amounts between evaluation areas were found to be large.

Therefore, an object of the present invention is to provide a technology for immobilizing peptides that allows a satisfactory amount of peptide to be immobilized on a solid phase support. In addition, another object of the present invention is to provide a technology for immobilizing peptides that allows a stabilized amount of peptide to be immobilized on a solid phase support. Moreover, another object of the present invention is to provide a technology for immobilizing peptides that allows the construction of a more highly reliable evaluation system.

As a result of conducting various studies on techniques for immobilizing peptides on a solid phase support in order to solve the above-mentioned problems, the inventors of the present invention obtained the finding that the immobilized amount of peptide can be improved and variations in the immobilized amount can be reduced by containing a surfactant such as sodium dodecyl sulfate in a peptide immobilization solution containing dissolved peptide therein. The inventors of the present invention completed the present invention on the basis of this finding. Namely, the following means are provided by the present invention:

(1) a peptide immobilization solution for immobilizing a peptide on a solid phase support, containing a surfactant;
(2) the solution described in (1), wherein the surfactant is a surfactant that is able to increase an immobilized amount of the peptide on the solid phase support in comparison with the use of a peptide immobilization solution not containing the surfactant
(3) the solution described hi (1) or (2), wherein the surfactant includes an ionic surfactant
(4) the solution described in any of (1) to (3), wherein the surfactant includes an anionic surfactant
(5) the solution described in (4), wherein the surfactant contains sodium dodecyl sulfate;
(6) the solution described in any of (1) to (5), further containing a salt
(7) the solution described in any of (1) to (6), wherein the peptide is a peptide composed of 50 or fewer amino acid residues;
(8) the solution described in any of (1) to (7), which is supplied to the solid phase support by a liquid droplet discharge method using piezoelectric driving or electrostatic driving;
(9) a method for supplying a peptide to a solid phase support, comprising:

a step of supplying the peptide to the solid phase support so that a state in which the peptide is present with a surfactant on the solid phase support is funned;

(10) the supply method described in (9), wherein the peptide supply step is a step of preparing a peptide immobilization solution containing the peptide and the surfactant, and supplying the peptide immobilization solution onto the solid phase support;
(11) the supply method described in (9) or (10), wherein the solid phase support is in the form of a plate, and

a step of supplying the peptide immobilization solution is a step of discharging the peptide immobilization solution onto the solid phase support as liquid droplets by a liquid droplet discharge method using piezoelectric driving or electrostatic driving

(12) a method for producing a peptide immobilized body in which a peptide is immobilized on a solid phase support comprising:

a step of supplying the peptide to the solid phase support so that a state in which the peptide is present with a surfactant on the solid phase support is formed; and

a step of immobilizing the peptide supplied to the solid phase support, on the solid phase support

(13) the production method described in (12), wherein the peptide supply step is a step of preparing one type or two or more types of peptide immobilization solutions containing the peptide and the surfactant, and supplying the peptide immobilization solution to the solid phase support;
(14) the production method described in (12) or (13), wherein the surfactant is an anionic surfactant;
(15) the production method described in (14), wherein the surfactant contains sodium dodecyl sulfate;
(16) the production method described in any of (12) to (15), wherein the peptide immobilization solution further contains a salt;
(17) the production method described in any of (12) to (16), wherein the peptide is a peptide composed of 50 or fewer amino acid residues;
(18) the production method described in any of (13) to (17), wherein the solid phase support is in the form of a plate, and

the peptide supply step is a step of discharging the peptide immobilization solution onto the solid phase support as liquid droplets by a liquid droplet discharge method using piezoelectric driving or electrostatic driving;

(19) a peptide immobilized body in which a peptide is immobilized on a solid phase support, which is obtained by the production method described in any of (12) to (18);
(20) a peptide array, comprising:

a plate-like solid phase support; and

two or more evaluation areas on the solid phase support that respectively retain one type or two or more types of peptides and one type or two or more types of surfactants;

(21) the peptide array described in (20), wherein the one type or two or more types of surfactants include an anionic surfactant; and
(22) the peptide may described in (20) or (21), wherein the one type of two or more types of peptides include a peptide composed of 50 or fewer amino acid residues.

### BRIEF DESCRIPTION OF THE DRAWINGS

FIG. 1 is a drawing showing a state in which a peptide immobilization solution of the present invention is formed by imparting onto a solid phase support;

FIG. 2 is a drawing schematically representing a peptide array produced in Example 1;

FIG. 3 is a drawing showing results of evaluating immobilized amounts of protein obtained in Example 1;

FIG. 4 is a drawing showing results of evaluating spot diameters obtained in Example 1;

FIG. 5 is a drawing schematically representing a peptide array produced in Example 2;

FIG. 6 is a drawing showing results of evaluating storage stability of a peptide array; and

FIG. 7 is a drawing showing results of evaluating variations in immobilized amounts of the same specimen in a peptide array.

### DESCRIPTION OF THE PREFERRED EMBODIMENTS

The present invention relates to a peptide immobilization solution and the use thereof. Namely, the present invention relates to a peptide immobilization solution, a method for supplying peptide onto a solid phase support, a method for producing a solid phase support on which peptide is retained, a solid phase support on which peptide is retained, a peptide array, and a method of using a peptide array.

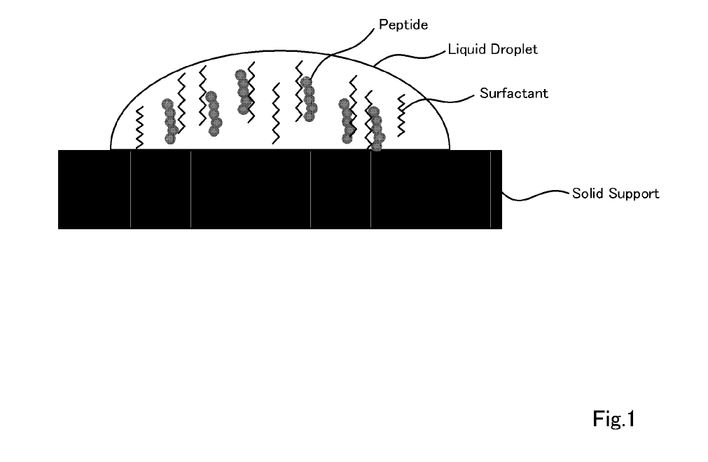

According to the peptide immobilization solution of the present invention, as shown in FIG. 1, when immobilizing a peptide on a solid phase support, a surfactant is also supplied onto the solid phase support together with the peptide to be immobilized. Namely, a state is formed in which the peptide and surfactant are both present on the solid phase support. When such a state is formed, the immobilized amount of peptide is increased and the immobilized amount of peptide is stabilized.

Although speculative and not intended to constrain the present invention, peptides have an electrical charge and polarity (non-polarity) corresponding to the amino acids of which they are composed. Surfactants having hydrophobic groups or hydrophilic groups adsorb and align peptides by interacting therewith, thereby creating an environment in which the peptides are discharged and immobilized on a solid phase support. This interaction between peptides and surfactants is presumed to contribute to immobilization of peptides on solid phase supports.

On the basis of the above, according to the peptide immobilization solution of the present invention, a satisfactory immobilized amount of peptide can be obtained on a solid phase support. In addition, according to the peptide immobilization solution of the present invention, a stable immobilized amount of peptide can be obtained on a solid phase support. Moreover, a more highly reliable evaluation system can be constructed.

Moreover, according to the peptide immobilization solution of the present invention, since a peptide is uniformly dissolved by surfactant and surface tension is decreased as a result of containing the surfactant, the peptide immobilization solution is suitable for a supply method in which it is discharged onto the solid phase support as liquid droplets by a liquid droplet discharge method.

In another aspect of the present invention, any of the above-mentioned effects can be obtained based on action resulting from peptide and surfactant both being present on a solid phase support. The following provides a detailed explanation of these embodiments of the present invention while suitably referring to the drawings. FIG. 1 shows a state in which the peptide immobilization solution of the present invention is supplied onto a solid phase support as liquid droplets.

Furthermore, in the present description, a “peptide” refers to a compound formed by the bonding of two or more amino acids by peptide bonds (—CO—NH—).

In addition, in the present description, “peptide immobilization” refers to immobilizing the above-mentioned “peptide” on a solid phase support by some form of interaction with the surface thereof. The interaction is not limited, and includes hydrogen bonding, dipole-dipole interaction, hydrophilic or hydrophobic interaction, ionic bonding, electrostatic bonding and covalent bonding.

In addition, in the present description, a “solid phase support” refers to an object having for at least a portion thereof a solid phase on which a peptide is immobilized. There are no particular limitations on the solid phase support of the present invention. There are also no limitations on the properties of the solid phase support

(Peptide Immobilization Solution)

The peptide immobilization solution is a solution for at least supplying a peptide onto a solid phase support for the ultimate purpose of immobilizing the peptide on the solid phase support. The peptide is present as a solute in the solution. The solvent of this solution is preferably an aqueous medium. The aqueous medium includes water and a mixture of water and an organic solvent compatible therewith. There are no particular limitations on the organic solvent, and example thereof is DMSO. Although suitably determined, the pH of the peptide immobilization solution can be about pH 4 to pH 10.

(Peptide)

The peptide to be immobilized in the peptide immobilization solution of the present invention may be a naturally-occurring peptide or synthetic peptide. Naturally-occurring peptides include those that exist in nature or fragments thereof. In addition, synthetic peptides may include, for example, those chemically synthesized by commonly known solid phase synthesis methods, and those synthesized using genetic engineering techniques. In addition, synthetic peptides may also be those synthesized by altering based on naturally-occurring peptides.

Examples of immobilized peptides include peptides having an amino acid sequence of a substrate recognition site of a prescribed enzyme, peptides having an amino acid sequence of a ligand recognition site that binds with a prescribed receptor, peptides having an amino acid sequence that serves as a binding site of a prescribed receptor or enzyme inhibitor, peptides bound by antibody or peptides having an amino acid sequence that serves as an epitope thereof, peptides having various physiological activities such as cytokines or hormones, and peptides having an amino acid sequence of an active site thereof. Furthermore, the immobilized peptide is only required to have the possibility of having an amino acid sequence as described above.

Although the immobilized peptide is not particularly required to separately retain a functional group that is crosslinked or condensed with a functional group on the surface of a solid phase support, such chemical modifications are not excluded. For example, although varying according to the type of functional group formed on the surface of the solid phase support, examples of such functional groups include cysteine or thiol groups and oxyamino groups. In addition, the immobilized peptide may be provided with an amino acid sequence that serves as a suitable linker. Linkers are suitably used in the case of binding to a solid phase support by chemical bonding and the like. A linker preferably has a functional group or modification required for chemical bond formation. Although there are no particular limitations on the number of amino acid residues that compose the linker, the overall length preferably does not exceed a length that is advantageous for immobilization.

Although there are no particular limitations on the number of amino acid residues that compose the immobilized peptide, in consideration of immobilization ability, the number of amino acid residues is preferably 50 or less, more preferably 30 or less, and even more preferably 20 or less and still more preferably 10 or less. In addition, although varying according to the type of interaction to be evaluated, the number of amino acid residues of the immobilized peptide is preferably 30 or less in consideration of difficulty of synthesis, specificity during screening, efficiency and the like. In addition, in consideration of searching for epitopes and the like, the number of amino acid residues is more preferably 20 or less. In addition, in consideration of the number of residues recognized by antibody as epitopes and the need for a so-called linker site to avoid steric hindrance, the number of amino acid residues is preferably 6 or more.

A single peptide immobilization solution may contain only one type of peptide or may contain two or more types of peptides. Only one type of peptide may be supplied and immobilized in a single evaluation area on a solid phase support to evaluate interaction between each peptide and a test sample, or two or more types of peptides may be supplied to a single evaluation area to evaluate interaction between two or more types of peptides and a test sample.

(Surfactant)

The peptide immobilization solution of the present invention can contain a surfactant. In the present invention, although the surfactant is at least required to be that which is able to solubilize a peptide to be immobilized, it is preferably a surfactant that is able to increase the immobilized amount of peptide on a solid phase support in comparison with the case of using a peptide immobilization solution not containing the surfactant. The use of such a surfactant makes it possible to increase the immobilized amount of peptide on a solid phase support.

Examples of surfactants include ionic surfactants, nonionic surfactants and amphoteric surfactants. In the present invention, ionic surfactants can be used preferably. Since peptides have a charge, it is thought that the surfactant also preferably be ionic in order to immobilize peptide. Among ionic surfactants, examples of anionic surfactants include fatty acid salts (RCOOM) such as sodium fatty acid salts, alkylbenzene sulfonates (RSO₃M) such as sodium alkylbenzene sulfonate, and monoalkyl sulfates (RSO₄M) such as sodium dodecyl sulfate (SDS) (in the above formulas, M represents an alkaline metal such as sodium). Anionic surfactants are preferable from the viewpoints of chemical stability and cost. SDS is more preferable from the viewpoint of having an extensive history of use in biomaterials. Examples of cationic surfactants include quaternary ammonium salts such as alkyl trimethyl ammonium salts, dialkyl dimethyl ammonium salts and alkylbenzyl dimethyl ammonium salts, as well as alkylpyridinium salts. Furthermore, the peptide immobilization solution may contain only one type of surfactant or may contain two or more types of surfactants.

In addition, since local hydrophilic groups and hydrophobic groups vary according to amino acid composition, in consideration of the orientation of the surfactant, amphoteric surfactants can also be used preferably. Examples of amphoteric surfactants include alkyl dimethyl amine oxides and alkyl carboxybetaines.

Moreover, nonionic surfactants can also be used preferably in consideration of resistant to changes in pH, low degree of bubbling, low level of irritation and work safety. Examples of nonionic surfactants include polyoxyethylene alkyl ethers, fatty acid sorbitan esters, alkyl polyglucosides, fatty acid diethanol amides and alkyl monoglyceryl ethers.

The peptide immobilization solution can further contain a salt. The salt is preferably that which does not impair the physiological activity of the peptide. Examples of such salts include phosphate salts and citrate salts having buffering ability. The type of buffering salt is suitably selected corresponding to the pH imparted to the peptide immobilization solution. The peptide immobilization solution can further contain other solutes as necessary.

(Solid Phase Support)

There are no particular limitations on the structure of the solid phase of the solid phase support to which the peptide immobilization solution is applied. The solid phase may be dense or porous having separate and/or continuous air bubbles. The solid phase support may be a knit body, woven body or entangled body comprising a combination of various forms of fibrous bodies. There are no limitations on the form of the solid phase support. Examples of forms of solid phase supports that can be employed include flat sheets, plates and spheres. In the case of forming peptides into an array, the solid phase support is preferably used in the form of a plate.

The surface on which peptide is immobilized is one surface of this type of solid phase support, and in the case of having a broad surface of a flat solid phase support, spherical surface or hollow portion, the surface on which peptide is immobilized may be the outer surface or inner surface thereof. There are no particular limitations on the material of these solid phase supports. Examples of materials that may be used include glass, ceramics, plastic, metal, wood and other natural materials.

In consideration of affinity with the peptide immobilization solution, the surface of the solid phase support on which peptide is immobilized is preferably hydrophilic. In addition, when using an ionic surfactant as a surfactant, the surface of the solid phase support preferably has ionic functional groups that are the opposite of the ions (anions and/or cations) possessed by the surfactant. Typically, this type of solid phase support itself has functional groups or such functional groups are imparted to the surface of the solid phase support. Although there are no particular limitations on these functional groups, examples of cationic functional groups that form cations when dissociated in water include amino groups such as primary or secondary amino groups and imino groups. In addition, examples of anionic functional groups that form anions when dissociated in water include carboxyl groups, phosphate groups and sulfonate groups.

In addition, the solid phase support can be provided with functional groups capable of bonding with peptide or separately added crosslinking agent on the surface thereof. Although there are no particular limitations on such functional groups, examples include active ester groups, epoxy groups, maleimido groups, formyl groups and benzylthioester groups.

The peptide immobilization solution as explained above can be supplied onto a solid phase support and form a state in which peptide and surfactant are both present on the solid phase support. As a result, the previously explained actions and the hie can be demonstrated. There are no particular limitations on the manner in which the peptide immobilization solution is supplied to the solid phase support. This is because the objective of this solution is to efficiently immobilize peptide on the solid phase support by forming a state in which peptide and surfactant are both present. This solution is preferably supplied to the solid phase support as liquid droplets. This is because peptides can be easily formed into an array when supplied in this manner Examples of apparatuses for realizing this form of supply include contact methods in which a pin contacts the solid phase support, and non-contact methods mediated by a liquid droplet discharge head used in ink jet applications and the like. In consideration of discharge accuracy and efficiency, a non-contact method mediated by a liquid droplet discharge head is more preferable. Even more preferably, a liquid droplet discharge head is used that employs piezoelectric driving or electrostatic driving. These driving methods are able to inhibit peptide denaturation while also suppressing the formation of air bubbles in the discharge flow path even in the case of a peptide immobilization solution containing surfactant. As a result, each droplet can be discharged with high accuracy, and a stable and large immobilized amount of peptide can be immobilized on the solid phase support. Thus, a peptide army can be easily formed that has a satisfactory immobilized amount of peptide and inhibits variations in the immobilized amounts of peptide between liquid droplets (evaluation areas). Since peptides demonstrate various properties according to their amino acid composition, a piezoelectric driving method is preferably employed due to its large driving force (discharge force) and large room for adjustment between each peptide.

(Supply of Peptide to Solid Phase Support)

The method for supplying peptide to the solid phase support of the present invention can be provided with a step of supplying a peptide to a solid phase support so that a state in which both surfactant and the peptide are present on the solid phase support is formed. The formation of such a state on the solid phase support facilitates immobilization of peptide on the solid phase support by enabling interaction between the peptide and the surfactant.

Liquid droplets containing surfactant and peptide are formed on the solid phase support, for example, in order to carry out the peptide supply step. In order to accomplish this, the peptide immobilization solution of the present invention containing peptide and surfactant can be prepared in advance, and this solution can be supplied as droplet onto the solid phase support. In addition, liquid droplets of peptide or surfactant may be supplied onto the solid phase support in advance, followed by supplying droplets of the other component of the peptide immobilization solution over the previously supplied component. The preliminarily imparted peptide or surfactant, may be in a state of liquid droplets containing peptide or surfactant at the point the other is subsequently supplied onto the solid phase support, or may be dried to a solid. The peptide immobilization solution of the present invention is preferably used in order to demonstrate the interactive effects of the peptide and surfactant

Furthermore, in order to form this type of solution on a solid phase support, the solid phase support is preferably in the form of a plate, and the peptide immobilization solution is preferably discharged onto the solid phase support as liquid droplets by a liquid droplet discharge method using piezoelectric driving of electrostatic driving.

(Production of Peptide Immobilized Body)

The method of the present invention for producing a solid phase support on which a peptide is retained can be provided with a step of supplying a peptide to a solid phase support so that a state in which the peptide is present on the solid phase support together with a surfactant is formed, and a step of immobilizing the peptide on the solid phase support. According to this method, the peptide is easily immobilized on the solid phase support due to interaction between the peptide and surfactant. As a result of immobilizing the peptide on the solid phase support while in this state, a larger amount of peptide is immobilized on the solid phase support. In addition, the peptide can be immobilized while suppressing variations in the immobilized amount thereof. The peptide supply step is as was previously explained in the section describing the method of the present invention for supplying peptide to the solid phase support

There are no particular limitations on the method for immobilizing the peptide on the solid phase support Peptide can be immobilized on the solid phase support by utilizing interaction between the peptide and the surface of the solid phase support and/or interaction between a surfactant and the surface of the solid phase support by having the peptide and surfactant both present in a suitable medium, and typically an aqueous medium, followed by distilling off the aqueous medium. In addition, peptide can also be immobilized on the solid phase support through covalent bonding by providing functional groups capable of crosslinking with the peptide and/or surface of the solid phase support, or by containing a crosslinking agent in the aqueous medium in addition to the peptide and surfactant. In the case of immobilizing the peptide accompanying the formation of covalent bonds, treatment such as heating of the solid phase support can be carried out as necessary. In an immobilization step such as heating, the surfactant is present with the peptide as is. After heating for a prescribed amount of time, a washing procedure such as removal of unreacted substances can be carried out as necessary. The surfactant may be substantially removed after carrying out the washing procedure.

A peptide array can be produced by using a solid phase support in the form of a plate and supplying the peptide immobilization solution and the like onto the solid phase support in liquid droplets. Although previously explained, the peptide immobilization solution is preferably discharged onto the solid phase support as liquid droplets by a liquid droplet discharge method using piezoelectric driving or electrostatic driving. Variations in the amount of liquid droplets supplied between liquid droplets (evaluation areas) can be inhibited, and as a result thereof, a peptide army can be produced in which variations in the immobilized amount of peptide are inhibited and the immobilized amount of peptide is satisfactory.

(Peptide Immobilized Body)

The peptide immobilized body obtained according to the production method of the present invention can have a configuration as described below. Namely, the peptide immobilized body can be provided with a solid phase support, and evaluation areas that respectively retain one type or two or more types of peptides and one type or two or more types of surfactants. The solid phase support is in a state prior to removal of the surfactant by washing and the like. The evaluation areas are areas prepared on the solid phase support for evaluating peptide. Peptides can be evaluated by immobilizing the peptide with the peptide and surfactant both present in the evaluation areas. A plate-like solid phase support is preferably formed by preparing these evaluation areas by arranging in the form of a matrix.

According to the peptide immobilized body of the present invention, effective amounts of peptide are retained in each evaluation area, and variations in the immobilized amount of peptide can be inhibited between evaluation areas. Consequently, a more highly reliable evaluation system can be easily constructed.

(Peptide Army)

The peptide array of the present invention can be provided with a plate-like solid phase support, and two or more evaluation areas on the solid phase support that respectively retain one type or two or more types of peptides and one type or two or more types of surfactants. In the peptide immobilized body of the present invention, the peptide array uses a plate shape for the shape of the solid phase support, and has a plurality, and preferably several tens or more, of evaluation areas prepared in the form of a matrix on the solid phase support. According to this type of peptide array, interactions having the possibility of being demonstrated by a large number of types of peptides can be evaluated highly reliably and efficiently.

In the peptide array of the present invention, the coefficient of variation of the immobilized amounts of peptide immobilized in each evaluation area formed on the solid phase support can be made to be an average of 20% or less, more preferably 10% or less and even more preferably 5% or less. A coefficient of variation of 5% or less enables the peptide army to be used as a clinical tool for quantitative analyses.

The peptide array of the present invention can be used to detect and evaluate various types of interactions between peptides and other substances. Since quantitatively effective amounts of peptide can be immobilized while inhibiting variations between each evaluation area, evaluations having satisfactory detection accuracy and reproducibility can be carried out, and interactions can be evaluated with high reliability. Although there are no particular limitations on interactions detected with the peptide array of the present invention, examples of arrays include substrate arrays of various enzymes such as protein kinase, protease or hydrolase, ligand arrays, inhibitor arrays, epitope arrays and other physiologically active peptide arrays.

Furthermore, each of the aspects explained in the sections on the peptide immobilization solution, peptide supply method and peptide immobilized body production method of the present invention, such as the immobilized peptide, surfactant, solid phase support or peptide supply method, are applied as is to the peptide immobilized body and peptide array of the present invention.

Although the following provides a detailed explanation of the present invention through examples thereof the present invention is not limited to the following examples.

### Example 1

### Production of Peptide Array Using Peptide Immobilization Solution Containing Surfactant

In the present example, a peptide array was produced using an ink jet type (piezoelectric drive type) of microarray production apparatus. In order to evaluate the viability of large-volume production, production of the array was carried out under the condition of the time from the start of spotting to completion being equivalent to the production of 2000 plates (and was carried out while suitably including trial runs). FITC-labeled peptides were used for the peptides to allow evaluation of the resulting array. The array was evaluated by carrying out peptide immobilization treatment followed by measuring the immobilized amount thereof with a fluorescence scanner. Furthermore, four types of peptides composed of the amino acid sequences shown in SEQ ID NO 1 to 4 were used for the peptides, and peptide immobilization solutions were prepared by dissolving the peptides in the solvent described below. Other peptide array production conditions were as indicated below.

| | |
|---|---|
| SEQ ID NO 1: | NQFLPYPYYAKPAAVR |
| SEQ ID NO 2: | STEVFTKKTKLTEEEK |
| SEQ ID NO 3: | EKNRLNFLKKISQRYQ |
| SEQ ID NO 4: | YQLDAYPSGAWYYVPL |

### (1) Peptide Immobilization Solution

Peptide: 16 residues, 4 types, 2.0 mg/ml

Solvent 0.1% by weight SDS, 20 mM phosphate buffer (pH 8.5)
The peptide immobilization solution was prepared by adding 0.2% by weight SDS solution to each type of powdered peptide, mixing, adding an equal amount of 40 mM phosphate buffer and confirming dissolution of peptide with a light microscope.
   (2) No. of spots: 1020 (255× four types)
   (3) Spot pattern: 15 rows×17 columns for each type of peptide (pitch: 200 μm)
   (4) Spotting speed: Conditioned on being equivalent to production of 2000 plates at the 1020th spot
   (5) Solid phase support: 762 mm×25.4 mm×1 mm (active ester plate)
   (6) Supply of peptide immobilization solution to solid phase support

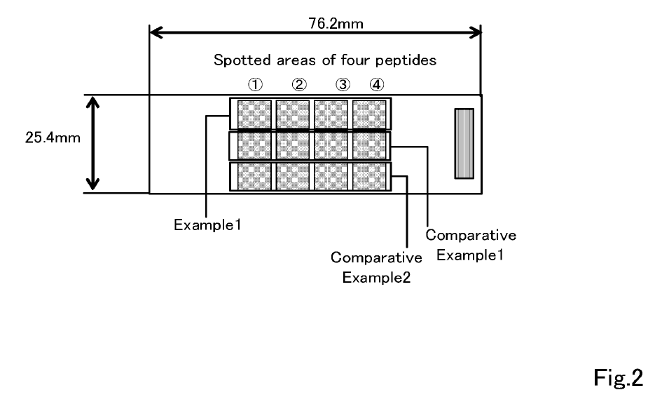

A prescribed amount of peptide solution was placed in an ink jet type of microarray production apparatus followed by spotting the peptide immobilization solution on the solid phase support under the conditions of (2) to (4) above. Furthermore, the spotted areas are shown in FIG. 2.

(7) Peptide immobilization

Peptides were immobilized on the solid phase support according to the procedure described below.

a) Heat treatment for 1 hour at 80° C.

b) Immersion for 15 minutes in (2×SSC, 0.2% SDS) solution (room temperature)

c) Immersion for 5 minutes in (2×SSC, 0.2% SDS) solution (95° C.)

d) Shaking about 10 times in sterilized water (3 times)

e) Centrifugal drying

### (8) Evaluation

Fluorescence intensity of the peptide array was measured with a fluorescence scanner (ArrayWorx, GE Healthcare Bio-sciences), and fluorescence intensity was expressed numerically with numerical analysis software (Gene Pix. Pro, Axon). Moreover, the amounts of immobilized peptide were evaluated based on fluorescence intensity, while variations in the immobilized amounts were evaluated based on the coefficient of variation (CV) of fluorescence intensity. In addition, CV were also separately calculated by measuring spot diameter (spot quantitative accuracy). Furthermore, CV was calculated as standard deviation/mean×100(%) (to apply similarly hereinafter).

Furthermore, peptide arrays were produced in Comparative Examples 1 to 3 under the same conditions as described above with the exception of the parameters described below.

### Comparative Example 1

### Different Solvent

(1) Peptide immobilization solution

Solvent: 5% by weight DMSO
5% by weight DMSO was added to each type of powdered peptide and mixed.

### Comparative Example 2

### Different Solvent

(1) Peptide immobilization solution

Solvent 20% by weight glycerol
20% by weight glycerol was added to each type of powdered peptide and mixed.

### Comparative Example 3

### Comparison of Spot Diameter

(1) Peptide immobilization solution

Solvent: 50% by weight DMSO
50% by weight DMSO was added to each type of powdered peptide and mixed.
   (2) No. of spots: 12 (3×4 types)
   (3) Spot pattern: 2 rows×6 columns
   (4) Spotting speed: Speed in accordance with apparatus performance (not conditioned on being equivalent to production of 2000 plates)
   (5) An apparatus icing spin method was used for the microarray production apparatus

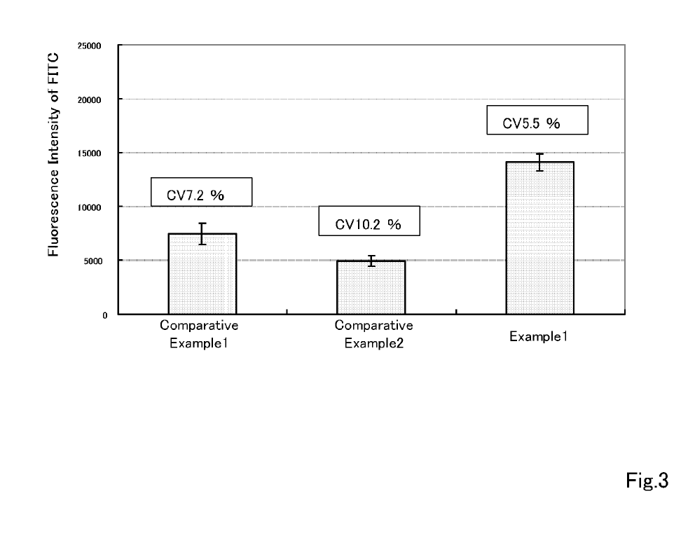

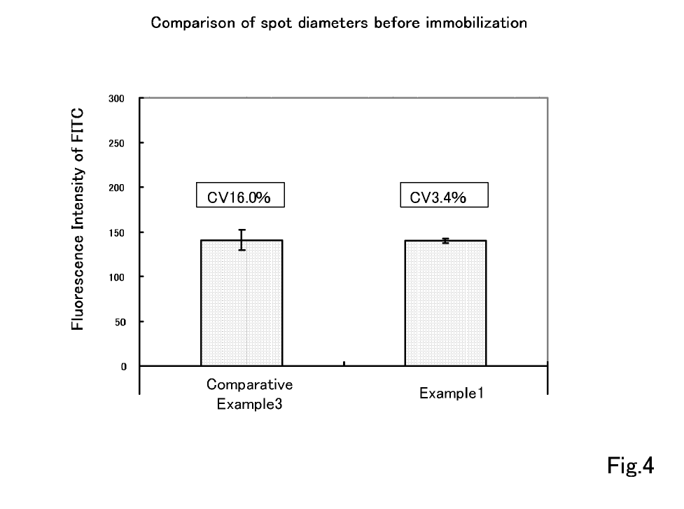

Results of immobilized amounts of peptide (fluorescence intensity) according to the type of solvent of the peptide immobilization solution based on the above-mentioned evaluation results (Example 1, Comparative Example 1 and Comparative Example 2) are shown in FIG. 3. In addition, results of comparing spot diameter according to differences in the method used to supply the peptide immobilization solution (ink jet method or pin method) (Example 1 and Comparative Example 3) are shown in FIG. 4.

In Example 1, Comparative Example 1 and Comparative Example 2, although four types of peptides were able to be dissolved in Example 1, there were peptides that were only able to be partially dissolved in Comparative Example 1 and Comparative Example 2. A comparison was made of fluorescence intensities (mean values) obtained for the peptide immobilization solutions of the peptide (SEQ ID NO 3: EKNRLNFLKKISQRYQ) that dissolved in all solvents in Example 1, Comparative Example 1 and Comparative Example 2. As shown in FIG. 3, fluorescence intensity obtained in Example 1 was two to three times higher than that of Comparative Example 1 and Comparative Example 2. In addition, the CV (%) of Example 1 was ¾ to ½ that of Comparative Example 1 and Comparative Example 2, respectively. On the basis of these results, the peptide immobilization solution of Example 1 was determined to be able to increase the immobilized amount of peptide as well as inhibit variations in immobilized amounts between spots.

In addition, as shown in FIG. 4, based on the results of comparing spot diameter between Example 1 and Comparative Example 3, in contrast to variations in spot diameter being extremely small in the case of the ink jet method, variations in spot diameter in the case of the pin method were about 5 times greater in terms of CV (%). On the basis of these results, liquid droplet discharge using the ink jet method was determined to allow the obtaining of stable spot diameter, and detection accuracy was also far superior to that of the pin method. In addition, liquid droplets were determined to be supplied to the solid phase support and retained thereon while inhibiting variations in droplet size by containing a surfactant in the solvent of the peptide immobilization solution.

In summary of the above results, the use of a surfactant as a peptide immobilization solution resulted in the advantages of a high peptide solubility, a large amount of immobilized peptide, and a low level of variation in the immobilized amount of peptide, and variations in the immobilized amount of peptide were determined to be small even under conditions premised on large-volume production. In addition, spots of greater quantitative accuracy were determined to be able to be obtained in comparison with conventional methods (pin-type arrays). On the basis of these results, the use of a surfactant in immobilization was determined to enable the production of high-quality peptide arrays applicable to large-volume production.

### Example 2

### Peptide Array Storage Stability

In the present example, an evaluation was made of the storage stability of the produced peptide arrays. In order to evaluate the storage stability of the peptide arrays, an assay was carried out using serum from patients allergic to milk. After producing the peptide arrays, the arrays were vacuum-packed, placed in a desiccator at room temperature, and stored for 2 weeks, 1 month, 3 months or 6 months, respectively. Assays were carried out using three peptide arrays each after each storage period had elapsed. Peptide arrays assayed on the same day in the absence of serum were used to determine the background level of non-specific adsorption for each peptide, and the mean value of that background level was subtracted from fluorescence intensity of each peptide on the actually assayed supports. The mean values of fluorescent intensities from which the background level had been subtracted were compared for each peptide based on their time-based changes and evaluated as storage stability of the peptide arrays. Furthermore, the peptides used in the present example included peptides associated with milk allergies for which there was the possibility of epitopes. The amino acid sequences of these peptides (SEQ ID NO 5 to 35) were as indicated below. In addition, peptide immobilization solutions were obtained by dissolving in the solvent shown below. Other conditions for preparing the peptide arrays were as shown below.

TABLE 1

| SEQ ID | Amino acid Sequence |
|---|---|
| 5 | SSEEIVPNSVEQKHIQ |
| 6 | HSMKEGIHAQQKEPMI |
| 7 | INPSKENLCSTFCKEV |
| 8 | QRYQKFALPQYLKTVY |
| 9 | ESPPEINTVQVTSTAV |
| 10 | QYTDAPSFSDIPNPIG |
| 11 | LEIVPNSAEERLHSMK |
| 12 | NLLRFFVAPFPEVFGK |
| 13 | LNEINQFYQKFPQYLQ |
| 14 | ESTEVFTKKTKLTEEE |
| 15 | EKNRLNFLKKISQRYQ |
| 16 | PLTQTPVVVPPFLQPE |
| 17 | VENLHLPLPLLQSWMH |
| 18 | RELEELNVPGEIVESL |
| 19 | HKEMPFPKYPVEPFTE |
| 20 | TQSLVYPFPGPIPNSL |
| 21 | HQPLPPTVMFPPQSVL |
| 22 | YIPIQYVLSRYPSYGL |
| 23 | NNQFLPYPYYAKPAAV |
| 24 | RCEKDERFFSDKIAKY |
| 25 | VRSPAQILQWQVLSNT |
| 26 | HPHLSFMAIPPKKNQD |
| 27 | TEAVESTVATLEDSPE |
| 28 | RELKDLKGYGGVSLPE |
| 29 | EQLTKCEVFRELKDLK |
| 30 | DIMCVKKILDKVGINY |
| 31 | TKIPAVFKIDALNENK |
| 32 | EVDDEALEKFDKALKA |
| 33 | FDKALKALPMHIRLSF |
| 34 | AQKKIIAEKTKIPAVF |
| 35 | YTDAPSFSDIPNPIGS |

(1) Peptide immobilization solution

Peptide: 16 residues, 31 types, 2.0 mg/ml

Solvent 0.1% by weight SDS, 20 mM phosphate buffer (pH 8.5)
Poly-DL-alanine (P9003, M.W.: 1000-5000, Sigma-Aldrich) was added to the 31 types of peptides as a negative control, and a total of 32 types of peptides were formed into an array.
The peptide immobilization solution was prepared by adding 0.2% by weight SDS solution to each type of powdered peptide, mixing, adding an equal amount of 40 mM phosphate buffer, and confirming peptide dissolution with a light microscope.
   (2) No. of spots: 192 (31 types×6 times)
   (3) Spot pattern: 12 rows×3 columns for each type of peptide (pitch: 200 μm)
   (4) Solid phase support: 76.2 mm×25.4 mm×1 mm (active ester plate)
   (5) Supply of peptide immobilization solution to solid phase support

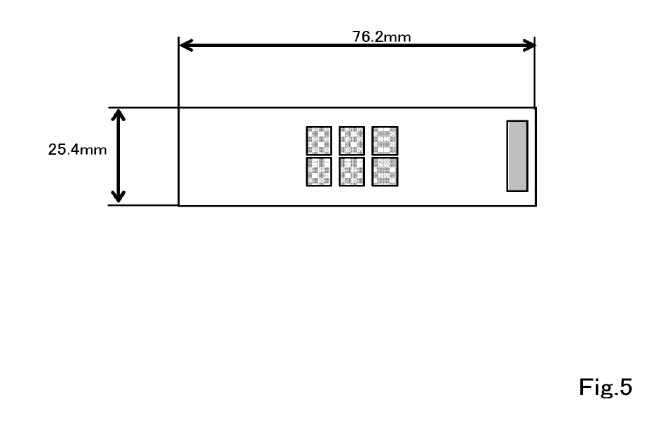

A prescribed amount of peptide solution was placed in an ink jet type (piezoelectric drive type) of microarray production apparatus, and the peptide immobilization solution was spotted onto the solid phase support under the conditions of (2) to (4) above. Furthermore, the spotted areas are shown in FIG. 5.

(6) Peptide immobilization

Peptides were immobilized on the solid phase support according to the following procedure for the peptide arrays after the storage periods had elapsed.

a) Heat treatment for 1 hour at 80° C.

b) Immersion for 15 minutes in (2×SSC, 0.2% SDS) solution (room temperature)

c) Immersion for 5 minutes in (2×SSC, 0.2% SDS) solution (95° C.)

d) Shaking about 10 times in sterilized water (3 times)

e) Centrifugal drying

### (7) Immunoassay

Immunoassays were =lied out according to the following procedure at completion of the storage periods.

a) Immersion for 90 minutes in 50 mM ethanolamine, 0.1% SDS and 0.1 M tris(hydroxymethyl)aminomethane solution (room temperature)

b) Immersion for 5 minutes in PBS-T (1×PBS, 0.1% Tween 20) solution (mom temperature, 3 times)

c) Support to which was applied 200 μL of patient serum diluted with 1% OVA, PBS-T solution (1:10) allowed to stand undisturbed in a humid chamber (by Sigma Corporation) at 37° C. for 1 hour after covering with a micro cover glass (Matsu ami Glass Ind. Co., Ltd., size: 24×60 mm, thickness: No. 4)

d) Support undergoing reaction in c) transferred to environment at 4° C. and allowed to stand undisturbed overnight

e) Micro cover glass removed in PBS-T solution

f) Immersion for 5 minutes in PBS-T solution (room temperature, 3 times)

g) Reacted using same procedure with 200 μL of goat anti-human IgE-Alexa 476 polyclonal antibodies diluted with 1% OVA, PBS-T solution (1:500) using the same procedure c) and allowed to stand undisturbed far 3 hours in a dark location (room temperature)

h) Micro cover glass removed in PBS-T solution

i) Immersion for 5 minutes in PBS-T solution (mom temperature, 3 times)

j) Shaking about 10 times in sterilized water (3 times)

### (8) Evaluation

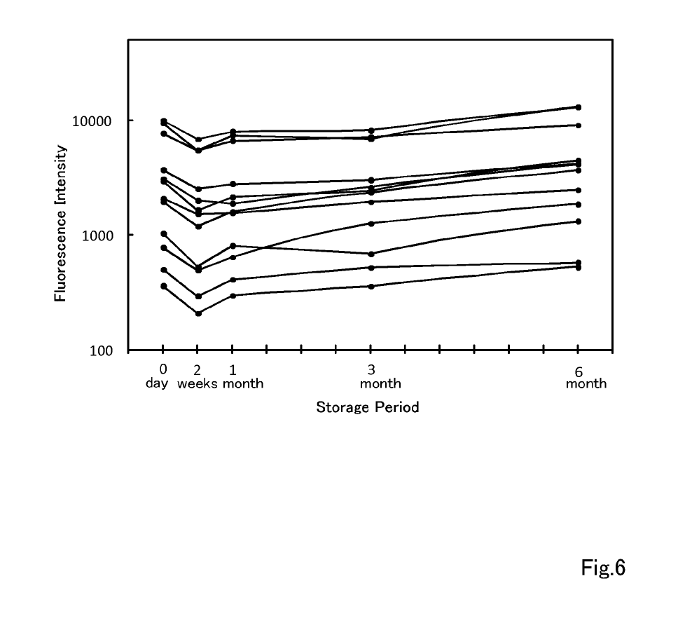

Fluorescence intensities of the peptide arrays were measured with a fluorescence scanner (Scanner Model GS2505B, Software G2565BA/DA, Agilent) and were expressed numerically with numerical analysis software (Gene Pix. Pro, Axon). Changes in the fluorescence intensity of each peptide of the assayed peptide arrays were compared to evaluate storage stability. The results are shown in FIG. 6.

As shown in FIG. 6, decreases in fluorescence intensity attributable to storage of the peptide arrays were not observed. In other words, the peptide arrays were determined to be able to adequately withstand room temperature storage in a desiccator following vacuum packing. On the basis of these Jesuits, the peptide arrays produced in the present example were determined to enable assays to be performed without incident for up to 6 months after production and demonstrate superior storage stability as a result of supplying a peptide immobilization solution to a solid phase support followed by storing in a vacuum.

### Example 3

### Evaluation of Accuracy of Produced Peptide Arrays in Immunoassay

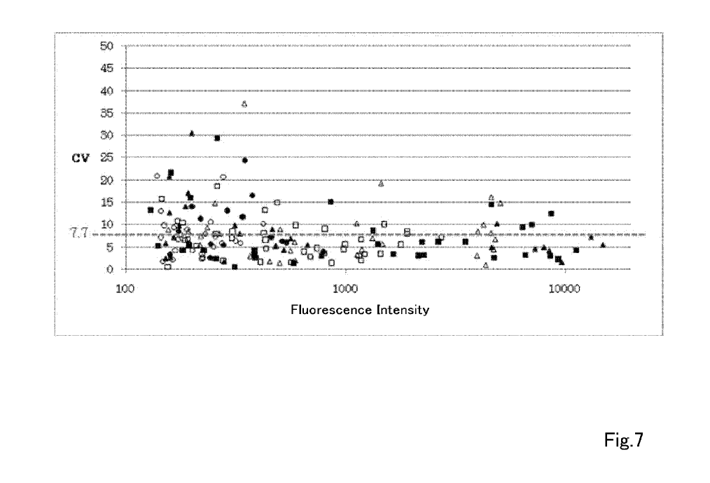

In the present example, the accuracy of the peptide arrays produced in Example 2 was determined in an assay. Thus, an assay was carried out using three specimens each of serum from patients allergic to milk and pooled serum from patients allergic to milk. Fluorescence intensity values of three spots for each peptide in the peptide arrays were measured for each serum, the coefficients of variation (CV) of these fluorescence intensity values were calculated, and the calculated values were used to evaluate accuracy of the produced peptide arrays with respect to assay. Furthermore, immunoassay and evaluation were carried out in the same manner as Example 2. The results are shown in FIG. 7: FIG. 7 was prepared by establishing a cutoff line at an S/N ratio of 2, and plotting CV values within the same peptide array (indicating the same solid phase support) of the fluorescence intensity of each peptide along with the mean values of fluorescence intensity.

Although signals for which low fluorescence intensity is detected generally tend to have a large CV in comparison with those having high fluorescence intensity, the peptide arrays produced in Example 2 exhibited a mean CV of about 7.7%. In consideration of a previous report by Tapia, et al. (Tapia, V., Bongartz, J., Schutkowski, M, Bruni, N., Weiser, A., Ay, B., Volkmer, R. and Or-Guil, M., Affinity profiling using the peptide microarray technology: a case study, Anal Biochem., 363, 108-118 (2007)) indicating an overall CV of about 28% among peptide array supports, the production of a more accurate peptide array is considered to have been realized.

On the basis of the above results, according to the present invention, it was determined that a high-quality peptide array can be produced that has high accuracy in terms of CV in comparison with existing peptide arrays obtained by an ink jet method (Tapia, et al.).

[Sequence Listing Free Text]

SEQ ID NO 1 to 35 Synthetic Peptides

[Sequence Listing]

1. A peptide immobilization solution for immobilizing a peptide on a solid phase support, containing a surfactant.

2. The solution according to claim 1, wherein the surfactant is a surfactant that is able to increase an immobilized amount of the peptide on the solid phase support in comparison with the use of a peptide immobilization solution not containing the surfactant.

3. The solution according to claim 1, wherein the surfactant includes an ionic surfactant.

4. The solution according to any of claims 1, wherein the surfactant includes an anionic surfactant.

5. The solution according to claim 4, wherein the surfactant contains sodium dodecyl sulfate.

6. The solution according to claim 1, further containing a salt.

7. The solution according to claim 1, wherein the peptide is a peptide composed of 50 or fewer amino acid residues.

8. The solution according to claim 1, which is supplied to the solid phase support by a liquid droplet discharge method using piezoelectric driving or electrostatic driving.

9. A method for supplying a peptide to a solid phase support, comprising the step of:
supplying the peptide to the solid phase support so that a state in which the peptide is present with a surfactant on the solid phase support is formed.

10. The supply method according to claim 9, wherein the peptide supply step is a step of preparing a peptide immobilization solution containing the peptide and the surfactant, and supplying the peptide immobilization solution onto the solid phase support.

11. The supply method according to claim 10, wherein the solid phase support is in the form of a plate, and
the step of supplying the peptide immobilization solution is a step of discharging the peptide immobilization solution onto the solid phase support as liquid droplets by a liquid droplet discharge method using piezoelectric driving or electrostatic driving.

12. A method for producing a peptide immobilized body in which a peptide is immobilized on a solid phase support, comprising the steps of:
supplying the peptide to the solid phase support so that a state in which the peptide is present with a surfactant on the solid phase support is formed; and
immobilizing the peptide supplied to the solid phase support, on the solid phase support.

13. The production method according to claim 12, wherein the peptide supply step is a step of preparing one type or two or more types of peptide immobilization solutions containing the peptide and the surfactant, and supplying the peptide immobilization solution to the solid phase support.

14. The production method according to claim 12, wherein the surfactant is an anionic surfactant.

15. The production method according to claim 14, wherein the surfactant contains sodium dodecyl sulfate.

16. The production method according to claim 12, wherein the peptide immobilization solution further contains a salt.

17. The production method according to claim 12, wherein the peptide is a peptide composed of 50 or fewer amino acid residues.

18. The production method according to claim 13, wherein the solid phase support is in the form of a plate, and
the peptide supply step is a step of discharging the peptide immobilization solution onto the solid phase support as liquid droplets by a liquid droplet discharge method using piezoelectric driving or electrostatic driving.

19. A peptide immobilized body in which a peptide is immobilized on a solid phase support, which is obtained by the production method according to claim 12.

20. A peptide array, comprising:
a plate-like solid phase support; and
two or more evaluation areas on the solid phase support that respectively retain one type or two or more types of peptides and one type or two or more types of surfactants.

21. The peptide array according to claim 20, wherein the one type or two or more types of surfactants include an anionic surfactant.

22. The peptide array according to claim 20, wherein the one type of two or more types of peptides include a peptide composed of 50 or fewer amino acid residues.
